(19) United States
(12) Patent Application Publication
(10) Pub. No.: US 2016/0058510 A1
(43) Pub. Date: Mar. 3, 2016

(54) SURGICAL PACKAGING AND METHODS OF USE

(71) Applicant: Howmedica Osteonics Corp., Mahwah, NJ (US)

(72) Inventors: Rebecca L. Blice, Akron, OH (US); Patrick Brown, Cleveland Heights, OH (US); Jason Tilk, Cleveland Heights, OH (US); Carolyn McNeeley, University Heights, OH (US)

(21) Appl. No.: 14/819,092

(22) Filed: Aug. 5, 2015

Related U.S. Application Data
(60) Provisional application No. 62/042,447, filed on Aug. 27, 2014.

(51) Int. Cl. A61B 19/02; B32B 3/06; B32B 27/32; B32B 7/14; B32B 27/06; B65D 81/24; B32B 5/18

(52) U.S. Cl. CPC A61B 19/026; B65D 81/245; B32B 3/06; B32B 5/18; B32B 7/14; B32B 27/065; B32B 27/32; A61B 2019/0268; A61B 2019/027; A61B 2019/0278; A61B 2019/0286; A61B 2019/4868

(57) ABSTRACT

A system for containing used surgical instruments, which includes an instrument container. The instrument container has outer and inner surfaces and a plurality of containment units disposed along the inner surface. The plurality of containment units each covers an area of the inner surface and each has restraints for holding at least one surgical instrument. The system also includes a decontaminant device having a foam sheet soaked with a disinfectant compound. The foam sheet has a surface area at least as large as the area of at least one of the plurality of containment units.

### CROSS-REFERENCE TO RELATED APPLICATIONS

The present application claims the benefit of the filing date of U.S. Provisional Patent Application No. 62/042,447, filed Aug. 27, 2014, the disclosure of which is hereby incorporated herein by reference.

### BACKGROUND OF THE INVENTION

Central Services Departments (“CSD”) are the central hub of healthcare facilities (e.g., hospitals, ambulatory surgery centers and the like), for receiving, processing, and distributing surgical supplies, including surgical instruments and implants used in orthopedic surgeries. In many of these healthcare facilities, numerous surgical procedures are performed each day. As such, large quantities of surgical instruments meant for different procedures and supplied by various sources pass through CSDs on a daily basis, where they may be decontaminated, assembled in sets, packaged, sterilized, stored and distributed when needed.

Some of the general types of surgical instruments that pass through CSDs are reusable, custom/patient specific and disposable instruments, all of which may be utilized in a single procedure. Unused reusable instruments typically arrive at the healthcare facility and are sent to the CSD to be sterilized and packaged for surgery. Once used, such instruments may then enter into a cycle of use where they may pass through a CSD on numerous occasions. Thus, it is common for custom and disposable instruments and implants to arrive separately to a CSD from the source and be processed separately from reusable instruments utilized in the same procedure.

While CSDs have proven to be beneficial since their inception for helping to provide sterile equipment to the operating room, their implementation and use has led to many drawbacks, particularly in an age where the number of surgical procedures and their associated costs are on the rise and where drug resistant microorganisms are frequent residents of hospitals and other healthcare facilities. One such drawback is that, due to the sheer volume and different types of instruments handled on a daily basis, there is an inevitable loss of such instruments each year. This consequently results in the loss of tens of thousands, if not hundreds of thousands, of dollars. Additionally, the separate processing of instruments utilized in the same procedure may result in the wrong instruments ending up in the operating room.

CSDs generally have varying procedures for handling unsterilized equipment particularly when such equipment is delivered from an outside source, which can result in a great deal of discrepancy in their state of sterility when they arrive in the operating room. Further, after surgery, some reusable instruments may be dumped in a tub, sprayed with an cleaning agent, and delivered to the CSD where they may sit for hours or days, which can result in corrosion, sometimes to a point where the instruments must be discarded leading to further monetary losses.

### BRIEF SUMMARY OF THE INVENTION

Disclosed herein are systems, devices and methods for packaging and handling surgical instruments. Such systems, devices, and methods, among other things, allow surgical instruments and implants to bypass a healthcare facility's CSD both when the instruments and implants are delivered to an operating room from an outside source and after the surgical procedure has concluded.

In one aspect of the present disclosure, a surgical instrument container includes first and second layers of a first material comprising inner and outer surfaces of the instrument container. Also included is an intermediate layer of a second material disposed between the first and second layer. A first and second containment unit is located along at least a portion of the inner surface and each has at least one restraint for holding at least one surgical instrument. At least one flap is located adjacent one of the containment units and is configured to fold over at least a portion of at least one of the containment units.

In addition, the first material may be a polymer material and the second material may be a woven material. More specifically, the first material may be one of polyethylene or polypropylene. The first and second containment units may be separated by a seam where folding the instrument container at the seam brings the first and second containment units together into a facing relationship. The first containment unit may include a first set of instruments for a first step in a surgical procedure, and the second containment unit may include a second set of instruments for a second step in a surgical procedure.

In another aspect of the present disclosure, a decontaminant device for use with surgical instruments includes a polymer sheet that has a first perimeter and inner and outer surfaces. The decontaminant device also includes a foam sheet that has a second perimeter smaller than the first perimeter and is coupled to the inner surface. The foam sheet is soaked in a preparation agent.

More specifically, the preparation agent may be one of a decontaminant compound or a moistening agent. Also, the inner surface may include first and second margin portions that are not covered by the foam sheet. The first and second margin portions may be located at opposite sides of the polymer sheet and may include adhesive thereon. In addition, the decontaminant device may have an open configuration and a closed configuration. When in the open configuration, the foam sheet may be exposed to an environment surrounding the decontaminant device, and when in the closed configuration the decontaminant device may be folded over itself and the first and second margin portions may be connected to the outer surface via the adhesive thereon such that the foam sheet is shielded from the environment.

In a further aspect of the present disclosure, a contamination container includes a first envelope. The first envelope includes an end and a first indicia of a first color. A second envelope includes an end coupled to the end of the first envelope and has a translucent second indicia of a second color. When the second envelope is pulled over the first envelope, the first and second indicia align to form a third indicia of a third color.

In addition, the first color may be maroon, the second color may be yellow, and the third color may be orange.

In a further aspect of the present disclosure, a system for containing used surgical instruments includes an instrument container. The instrument container has outer and inner surfaces and a plurality of containment units disposed along the inner surface. The plurality of containment units each cover an area of the inner surface and each have a restraint for holding at least one surgical instrument. Also included in the system is a decontaminant device that includes a foam sheet soaked with a disinfectant compound. The foam sheet has a surface area at least as large as the area of at least one of the plurality of containment units.

In addition, the system may include a contamination container that has a first and second envelope coupled together at respective ends. The first envelope may have a first indicia of a first color and the second envelope may have a second indicia of a second color. When the second envelope is pulled over the first envelope, the first and second indicia may align to form a third indicia of a third color. Each envelope may have a volume sufficiently large to encapsulate the surgical instrument container and decontaminant device.

Continuing with this aspect, the instrument container may include first and second flaps each disposed on opposite sides of the containment units and may be configured to fold over the instruments and decontaminant device when the decontaminant device is laid over the plurality of containment units. The decontaminant device may also include a polymer sheet that has a first perimeter and an inner surface. The foam sheet may have a second perimeter smaller than the first perimeter and may be coupled to the inner surface of the polymer sheet. 18. The inner surface may also include first and second margin portions that are not covered by the foam sheet. The first and second margin portions may be located at opposite sides of the polymer sheet and including an adhesive thereon. The decontaminant device may have an open configuration and a closed configuration. When in the open configuration, the foam sheet may be exposed to an environment surrounding the decontaminant device, and when in the closed configuration the decontaminant device may be folded over itself and the first and second margin portions may be connected to the outer surface via the adhesive thereon such that the foam sheet is shielded from the environment.

In addition, the instrument container may include a first and second layer of polymer material that comprise the inner and outer surfaces of the instrument container, and an intermediate layer of woven or nonwoven material disposed between the first and second layer. The first and second containment units may be separated by a seam, and wherein folding the instrument container at the seam brings the first and second containment units together into a facing relationship. The first containment unit may include a first set of instruments for a first step in a surgical procedure, and the second containment unit may include a second set of instruments for a second step in a surgical procedure. The instrument container may be a plurality of instrument containers each made from a polymer material of a different color indicating a particular set of instruments contained therein.

### BRIEF DESCRIPTION OF THE DRAWINGS

The features, aspects, and advantages of the present invention will become better understood with regard to the following description, appended claims, and accompanying drawings in which:

FIG. 1A shows a plurality of instrument containers in an open configuration and containing surgical instruments in accordance with the present invention.

FIG. 1B shows the instrument containers of FIG. 1A in a closed configuration.

FIG. 1C shows one embodiment method of packaging the instrument containers of FIG. 1A.

FIG. 2 shows one embodiment of a return kit containing a decontaminant pad.

FIG. 3 shows the decontaminant pad of FIG. 2.

FIG. 4A shows one embodiment of a biohazard container.

FIGS. 4B and 4C shows one embodiment method of using the contamination container of FIG. 4A.

### DETAILED DESCRIPTION

As used herein, the term “sterile” means substantially free from microorganisms, and the term “clean” means non-sterile and generally free from dirt, contaminants and other clinging particulates so as to help minimize transmission of microorganisms. Also, as used herein, the terms “about,” “generally” and “substantially” are intended to mean that slight deviations from absolute are included within the scope of the term so modified.

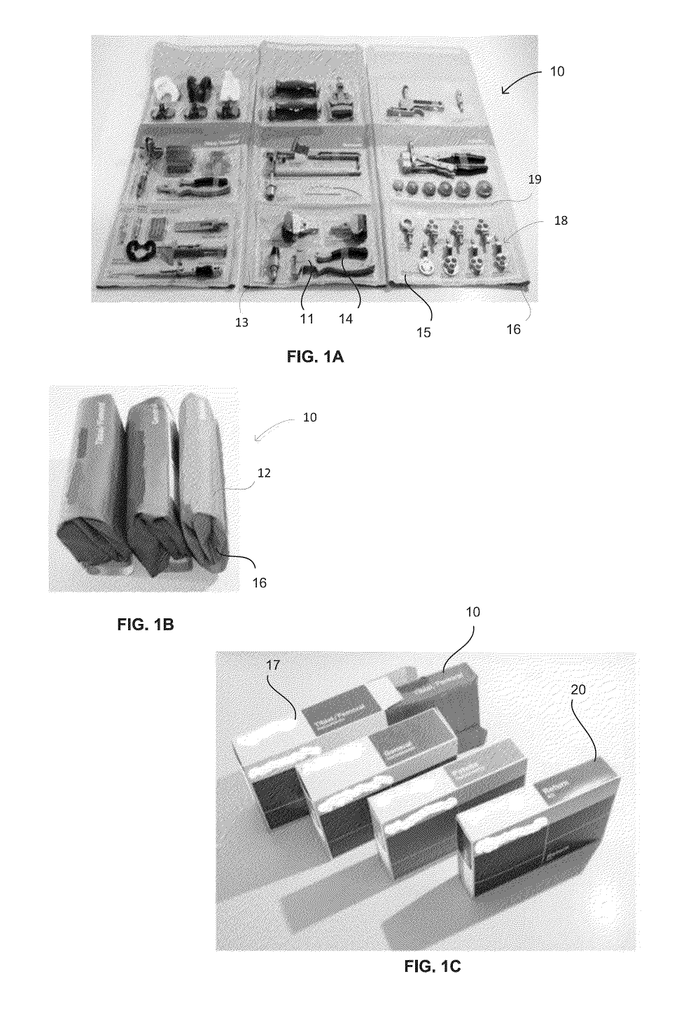

FIG. 1A depicts one embodiment of an instrument container 10 containing a plurality of surgical instruments 11. Instrument container 10 is a flexible wrap formed from multiple layers of a polymer material and a woven or non-woven fabric. The polymer material, such as polyethylene, polypropylene, and the like, is preferably a liquid repelling material and capable of being sterilized. The polymer material and woven/non-woven material are sandwiched together and connected by heat sealing, radio frequency welding, ultrasonic welding, stitching, adhesive bonding, or the like. The polymer material preferably comprises outer surface 12 and inner surface 13 of instrument container 10 to help prevent liquid, such as blood, from being absorbed therein. The woven/nonwoven material is preferably disposed between inner and outer surfaces 12, 13 of polymer material to help provide padding for instruments 11.

An additional layer of transparent material, such as a transparent polymer, may be connected to inner surface 13 of instrument container 10, such as by the techniques described above. This transparent material may be cut in strategic locations to provide restraining straps 14 for individual surgical instruments 11 in order to help restrain instruments from movement during transport and to help organize instruments 11 for surgery. The underlying polymer material can be screen-printed to include instrument information 15, such as worded descriptions or shadow boxes having the general shape of a particular instrument, in order to help identify the particular instrument restrained in a particular strap or straps, and also help identify which instruments should be placed within straps 14 in order to ensure all instruments 11 are included in the kit.

Where multiple instrument containers are provided, each container 10 may be color coded to help identify the type of surgical instruments contained therein. In one example, surgical instruments for a total knee arthroplasty may be contained within multiple instrument containers, wherein the tibial and femoral instruments may be contained in a green instrument container, the patellar instruments may be contained in an orange instrument container, and the universal instruments may be contained in a blue instrument container.

Each instrument container 10 preferably includes at least two containment units 18 in which the instruments may be contained in further subdivisions. For example, in an instrument container containing patellar instruments, the container 10 may be divided into three containment units 18 in which a first containment unit includes drilling templates, a second containment unit includes a plurality of trials, and a third containment unit includes measuring devices and/or cutting instruments.

In one embodiment, instrument container 10 may contain two or more containment units 18 where each containment unit 18 coincides with a different step in the surgical procedure relating to a particular anatomic feature. In such an embodiment, each containment unit may be color coded or numbered to indicate where such instruments are to be used in the surgical flow. In another embodiment, instrument container 10 may include two or more containment units 18 containing size specific instruments and at least one containment unit 18 including size independent instruments. Each containment unit 18 is preferably separated by a seam 19 or unfilled space such that each containment unit can be folded over each other at the seams 19 in order to provide a reduced profile of container 10 during transport and in order to help maintain a sterile environment therein.

Each instrument container 10 also includes flaps 16 flanking the containment units 18 that can be folded over instruments 11 to help prevent contaminants from entering into and escaping from instrument container 10. When instruments 11 are laid out for surgery, instrument container 10 can be placed on an instrument tray or cart (not shown) and act as a sterile barrier or an additional sterile barrier between the cart/tray and instruments 11. Additionally, flaps 16 can drape over the sides of the cart/tray to help prevent instrument container 10 from falling off the cart/tray.

In a method of preparing or packaging surgical instruments 11 for shipment, instruments 11 are placed into their associated restraining straps 14 while instrument container 10 is in an open configuration, as best shown in FIG. 1A. With instruments 11 restrained by straps 14, instrument container 10 is then closed into a closed configuration by folding flaps 16 over instruments 11 and folding containment units 18 over each other at seams 19, which helps lower the profile of instrument container 10 for shipping and handling, as best shown in FIG. 1B. Instrument container 10 may be held closed by fabric hook and loop fasteners, metallic hook and loop fasteners, buttons, snaps, or the like.

Instrument container 10 and instruments 11 contained therein are then placed into a box 17 (best shown in FIG. 1C), or some other protective container, such as a polymer or fabric bag. In one embodiment, instrument container 10 is placed into sterile packaging, which may include, but is not limited to, sterilization containers, such as sterilization bags, shrink wrap containers, and vacuum containers. In one example, instrument container 10 may be placed into a first sterilization container, sealed and then placed into a second sterilization container and sealed. The double contained instrument container 10 is then put into box 17 to help protect the integrity of the double barrier, and then shrink wrapped or vacuum sealed in a vacuum container. In another example, instrument container 10 containing instruments 11 may be shrink wrapped in a shrink wrap container or vacuum sealed in a vacuum container and, optionally, placed into box 17.

In some embodiments, instruments 11 may be placed into sterile packaging prior to being placed into instrument container 10. In one example, each instrument 11 may be placed in a sterilization container and placed into restraining straps 14. In another example, each instrument 11 may be shrink wrapped and placed into a respective restraining strap 14.

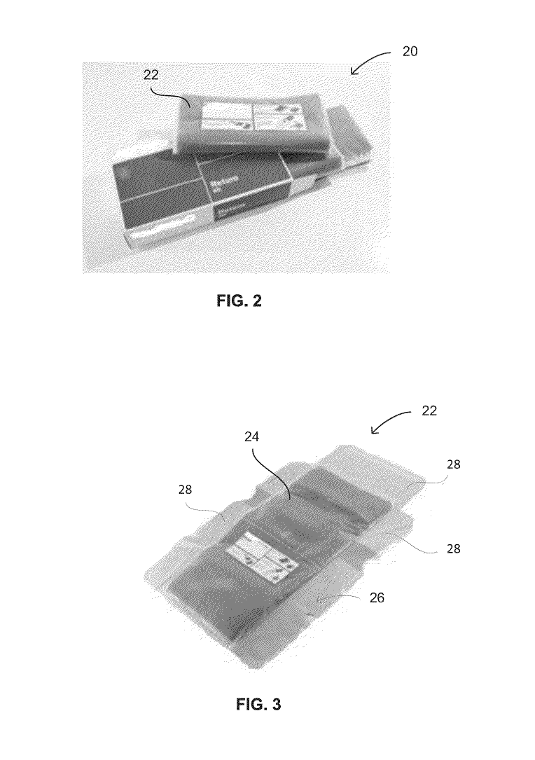

After the surgical procedure has been performed, instruments 11 can be placed back into instrument container 10 in a constrained or unconstrained fashion for shipment back to the source. This may be facilitated by a return kit 20, which is depicted in FIGS. 1C and 2. Return kit 20 includes decontaminant pads 22 and contamination containers 30. In another embodiment, return kit 20 may only include contamination containers 30 or may include contamination containers 30 and other instrument preparation materials in lieu of or in addition to decontaminant pads 22. Examples of instrument preparation materials other than decontaminant pads 22 may include disinfectant wipes and/or cleaning agents, such as enzymatic cleaners, and/or moistening agents such as distilled water to wipe down the instruments while still in a moist state to help make the cleaning process easier once returned to the source. In other embodiments, a return kit may contain other items such as cloths or soft barriers that may be wrapped around each instrument to help prevent damage during transport.

As shown in FIG. 3, decontaminant pad 22 includes a foam, or otherwise padded, sheet 24, that may be open celled or have perforations throughout. Foam sheet 24 is attached to a polymer sheet 26, such as a sheet of polypropylene, so that one side of foam sheet 24 is exposed and an opposite side is covered by polymer sheet 26. Polymer sheet 26 preferably extends beyond the periphery of foam sheet 24 to form margin portions 28 that may include pressure sensitive adhesive. The foam sheet 24 is generally rectangular in shape and preferably has a surface area sufficiently large to cover each containment unit 18 of an instrument container 10. In another embodiment, a foam sheet 24 may have a surface area substantially equal to or larger than one of the containment units 18. In such an embodiment, several decontaminant pads 22 may be deployed to cover the used surgical instruments 11.

Foam sheet 24 is preferably soaked with a decontaminant agent, such as a disinfecting enzymatic liquid that may include bactericidal, fungicidal, virucidal, and/or tuberculocidal properties, for example. Alternatively, foam sheet 24 may be soaked in a moistening agent, such as distilled water. Disinfectant pad 22 is folded over itself and sealed with the pressure sensitive adhesive. The soaked foam sheet is kept moist and prevented from leaking by polymer sheet 26. In one embodiment, the exposed side of foam sheet 24 may be covered with a peel away cover such that when the cover is peeled away foam sheet 24 and polymer sheet 26 remain. In another embodiment, the peel away cover may peel away from both sides of the foam sheet leaving only foam sheet 24 for application to surgical instruments 11.

In a method of using decontaminant pad 22, after instruments 11 have been placed back into instrument container 10 once the surgical procedure has concluded, decontaminant pad 22 is opened and placed over the used instruments 11 such that foam sheet 24 is exposed to instruments 11. Flaps 16 of instrument container 10 are folded over instruments 11 and decontaminant pad 22 and then folded over itself helping to contain the instruments, pad, decontaminant agent, and any contaminating agents therein in the closed configuration. In one embodiment, each flap 16 of the instrument container 10 is large enough to completely reach across the containment units 18 when instruments 11 and decontaminant pad 22 are located on instrument container 10. In another embodiment, flaps 16 are sufficiently large to overlap each other, while being sufficiently small so as to not be able to completely reach across the containment units 18 while instruments 11 and decontaminant pad 22 are located on instrument container 10.

Thereafter, instrument container 10 is placed back into box 17, which is then placed into contamination container 30. In an alternative method embodiment, in lieu of decontaminant pad 22 or in conjunction with decontaminant pad 22, disinfectant wipes (not shown) that are moist with the disinfectant agent may be used to wipe down instruments 11 prior to being placed back into instrument container 10.

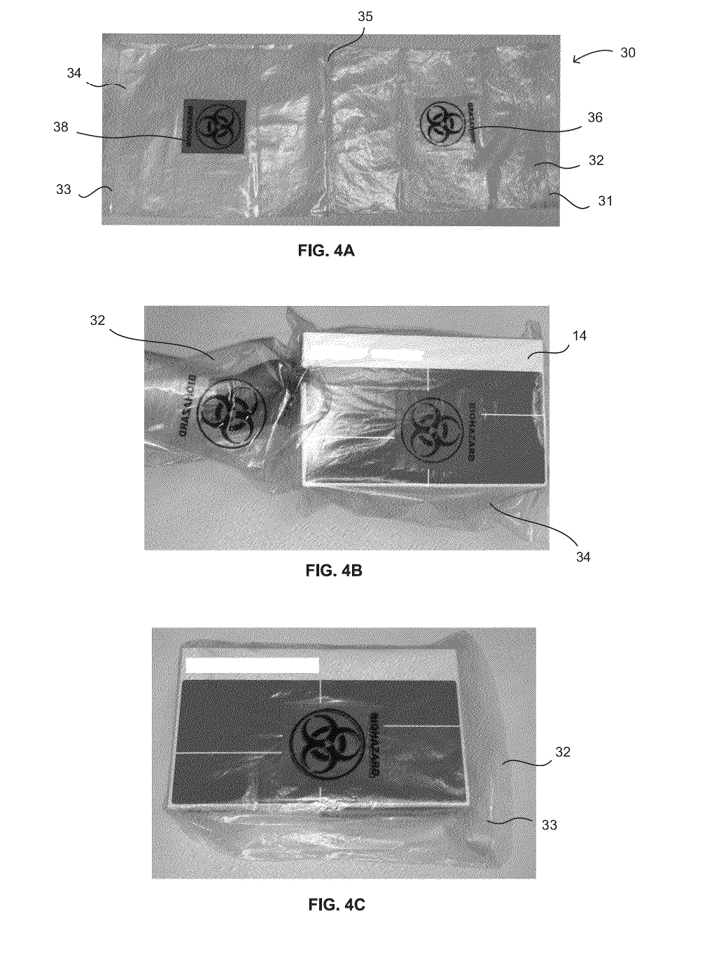

FIGS. 4A-C depict contamination container 30, which includes a first and second envelope 32, 34 preferably formed from a polymer material, such as polypropylene or polyethylene. First and second envelopes 32 and 34 each have a re-sealable end 31, 33 and a closed end. Re-sealable ends 31, 33 may be re-sealable by a zipper, adhesive, or the like. In one embodiment, resalable ends 31, 33 may be sealable only once. The closed ends are coupled together to form a barrier between the insides of each envelope 32, 34. First envelope 32 includes a first contamination marker 36 of a first color, and second envelope 34 includes a second contamination marker 38 of a second color. When used, contamination markers 36 and 38 overlap each other to form a third color, indicating that contaminated material is contained within the envelopes 32, 34. For example, the first color may be translucent yellow, the second color may be opaque maroon, and the third color may be orange.

In one method of using contamination container 30, after decontaminant pad 22 is applied to instruments 11 and instrument container 10 is placed back within box 17, box 17 is placed into second envelope 34, as best shown in FIG. 4B. Second sealable end 33 of second envelope 34 is sealed, and first envelope 32 is pulled over second envelope 34 and box 17 such that first envelope 32 inverts and first contamination marker 36 overlaps second contamination marker 38, as best shown in FIG. 4C. First re-sealable end 31 is sealed and the contamination markers interact to form the third color to indicate contaminated items are contained therein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A surgical instrument container, comprising:
first and second layers of a first material comprising inner and outer surfaces of the instrument container;
an intermediate layer of a second material disposed between the first and second layer;
a first and second containment unit located along at least a portion of the inner surface and each having at least one restraint for holding at least one surgical instrument; and
at least one flap located adjacent one of the containment units and being configured to fold over at least a portion of at least one of the containment units.

2. The container of claim 1, wherein the first material is a polymer material and the second material is a woven material.

3. The container of claim 2, wherein the first material is one of polyethylene and polypropylene.

4. The container of claim 1, wherein the first and second containment units are separated by a seam, and wherein folding the instrument container at the seam brings the first and second containment units together into a facing relationship.

5. The container of claim 4, wherein the first containment unit includes a first set of instruments for a first step in a surgical procedure, and the second containment unit includes a second set of instruments for a second step in a surgical procedure.

6. A decontaminant device for use with surgical instruments, comprising:
a polymer sheet having a first perimeter and inner and outer surfaces; and
a foam sheet having a second perimeter smaller than the first perimeter and being coupled to the inner surface, the foam sheet being soaked in a preparation agent.

7. The decontaminant device of claim 6, wherein the inner surface includes first and second margin portions that are not covered by the foam sheet, the first and second margin portions being located at opposite sides of the polymer sheet and including adhesive thereon.

8. The decontaminant device of claim 7, wherein the decontaminant device has an open configuration and a closed configuration wherein:
when in the open configuration, the foam sheet is exposed to an environment surrounding the decontaminant device, and
when in the closed configuration the decontaminant device is folded over itself and the first and second margin portions are connected to the outer surface via the adhesive thereon such that the foam sheet is shielded from the environment.

9. The decontaminant device of claim 6, wherein the preparation agent is one of a decontaminant compound or a moistening agent.

10. A system for containing used surgical instruments, comprising:
an instrument container having outer and inner surfaces and a plurality of containment units disposed along the inner surface, the plurality of containment units each covering an area of the inner surface and each having a restraint for holding at least one surgical instrument; and
a decontaminant device having a foam sheet soaked with a disinfectant compound, the foam sheet having a surface area at least as large as the area of at least one of the plurality of containment units.

11. The system of claim 10, further comprising a contamination container having a first and second envelope coupled together at respective ends.

12. The system of claim 11, wherein the first envelope has first indicia of a first color, the second envelope has a second indicia of a second color, and wherein when the second envelope is pulled over the first envelope the first and second indicia align to form a third indicia of a third color.

13. The system of claim 12, wherein each envelope has a volume sufficiently large to encapsulate the surgical instrument container and decontaminant device.

14. The system of claim 10, wherein the instrument container includes first and second flaps each disposed on opposite sides of the containment units and configured to fold over the instruments and decontaminant device when the decontaminant device is laid over the plurality of containment units.

15. The system of claim 10, wherein the decontaminant device further includes a polymer sheet having a first perimeter and an inner surface, and the foam sheet has a second perimeter smaller than the first perimeter and is coupled to the inner surface of the polymer sheet.

16. The system of claim 15, wherein the inner surface includes first and second margin portions that are not covered by the foam sheet, the first and second margin portions being located at opposite sides of the polymer sheet and including an adhesive thereon.

17. The system of claim 16, wherein the decontaminant device has an open configuration and a closed configuration wherein:
when in the open configuration, the foam sheet is exposed to an environment surrounding the decontaminant device, and
when in the closed configuration the decontaminant device is folded over itself and the first and second margin portions are connected to the outer surface via the adhesive thereon such that the foam sheet is shielded from the environment.

18. The system of claim 10, wherein the instrument container includes a first and second layer of polymer material comprising the inner and outer surfaces of the instrument container, and an intermediate layer of woven or nonwoven material disposed between the first and second layer.

19. The system of claim 18, wherein the first and second containment units are separated by a seam, and wherein folding the instrument container at the seam brings the first and second containment units together into a facing relationship.

20. The system of claim 10, wherein the instrument container is a plurality of instrument containers each being made from a polymer material of a different color indicating a particular set of instruments contained therein.
